(19) United States
(12) Patent Application Publication
(10) Pub. No.: US 2024/0052362 A1
(43) Pub. Date: Feb. 15, 2024

(54) LETTUCE PLANT RESISTANT TO DOWNY MILDEW AND RESISTANCE GENE

(71) Applicant: Enza Zaden Beheer B.V., Enkhuizen (NL)

(72) Inventors: Bas Ter Riet, Enkhuizen (NL); Mathieu André Pel, Enkhuizen (NL); Tieme Zeilmaker, Enkhuizen (NL); Judit Nadal Bigas, Enkhuizen (NL)

(21) Appl. No.: 18/268,066

(22) PCT Filed: Oct. 29, 2021
(86) PCT No.: PCT/EP21/80116

§ 371 Date: Jun. 16, 2023

(30) Foreign Application Priority Data
Dec. 18, 2020 (WO) PCT/EP2020/087264

(51) Int. Cl. C12N 15/82; A01H 6/14; A01H 1/04; C07K 14/415

(52) U.S. Cl. CPC C12N 15/8282; A01H 6/1472; A01H 1/045; C07K 14/415

(57) ABSTRACT

The present invention relates to a lettuce plant that is resistant to downy mildew, more specifically to a lettuce plant that comprises a mutated gene that confers broad spectrum resistance to oomycetes in lettuce, more specifically B. lactucae. Furthermore, the present invention relates to a resistance gene and a method for obtaining a lettuce plant that is resistant to downy mildew, wherein the method comprises the step of mutating a gene.

The present invention relates to a lettuce plant that is resistant to downy mildew, more specifically to a lettuce plant that comprises a resistance gene that confers broad spectrum resistance to Bremia lactucae in lettuce. Furthermore, the present invention relates a resistance gene and a method for providing a lettuce plant that is resistant to downy mildew.

Downy mildew refers to several types of oomycete microbes that are pathogens of plants. Downy mildew can originate from various species, but mainly of Peronospora, Plasmopara and Bremia. Downy mildew is a problem in many food crops, for example in lettuce caused by B. lactucae, affecting the production of this crop worldwide. Plants that are being affected include food crops such as brassicas (e.g. cabbage), grape, spinach, lettuce, onion, and cucumber. Downy mildew infection shows symptoms of discoloured areas on upper leaf surfaces in combination with white, grey or purple mould located on the lower side of the leaf facing the floor. Disease is spread from plant to plant by airborne spores.

Lettuce, mostly known as Lactuca sativa, but also including Lactuca species such as L. serriola, L. saligna or L. virosa, is a very important crop worldwide. Some of the most popular varieties available are Iceberg, Romaine, Butterhead, Batavia and Oakleaf. There are many plant pathogens that affect L. sativa, and some of the diseases caused by these pathogens are downy mildew, sclerotinia rot, powdery mildew, fusarium wilt of which the most important disease is lettuce downy mildew, which is caused by the B. lactucae, an oomycete pathogen that belong to Peronosporaceae.

For some vegetable crops, such as lettuce, cultivars with resistance to downy mildew are available. However, the pathogen under pressure will mutate to break down the disease resistance and new disease resistance in crops is needed to control infection. Especially in lettuce the occurrence of downy mildew resistance is particularly complex as there are many different races, and new downy mildew resistant species emerging all the time, as found in European and the USA markets.

In lettuce, infection of B. lactucae result in yellow to pale green lesions that eventually become necrotic due to secondary pathogens leading to major crop losses. Fungicides can be used to control B. lactucae, but eventually B. lactucae becomes immune to these chemicals, because over time the pathogen also acquires resistance to fungicides. Furthermore, there are multiple lettuce varieties available that are resistant to B. lactucae but resistance is quickly overcome because new Bremia races develop rapidly. Therefore, it is of the utmost importance to find other methods to control B. lactucae infection. Most preferably is to identify a resistance gene that gives broad resistance against B. lactucae and to provide for lettuce plants that are resistant to downy mildew. Therefore, identification of resistance genes is a promising alternative.

Considering the above, there is a need in the art for to provide plants that are resistant to downy mildew and wherein plants have a broad-spectrum resistance against this pathogen. Furthermore, it is an object of present invention to provide a method to obtain such downy mildew resistant plants.

It is an object of the present invention, amongst other objects, to address the above need in the art. The object of present invention, amongst other objects, is met by the present invention as outlined in the appended claims.

Specifically, the above object, amongst other objects, is met, according to a first aspect, by the present invention by a downy mildew resistant lettuce plant, wherein said lettuce plant comprises a SE17 resistance gene encoding a protein comprising a resistance domain 1 represented by amino acid sequence of SEQ ID No. 2 and resistance domain 2 which is represented by the amino acid sequence of SEQ ID No. 6, wherein downy mildew resistance is provided by one or more mutations in resistance domain 1 and/or resistance domain 2 and wherein said lettuce plant is resistant to Bremia lactucae races B1:12 to B1:36. The downy mildew resistance conferring gene SE17 is a dominant resistance trait, and may be homozygous or heterozygous present in a downy mildew resistant lettuce plant. For the first time a resistance gene against B. lactucae has been found in a lettuce plant that is located on chromosome 2 besides Dm3 in the MRC2 (major resistance cluster 2) that can be linked to plant disease resistance. This SE17 resistance gene of the present invention gives resistance to B. lactucae races B1:16 to B1:36, and also US strains B1:1 to B1:9. Furthermore, disease resistance test show that the SE17 resistance gene further provides resistance to Bremia races B1:2, B1:4, B1:5, B1:10, and B1:12 to B1:15. It is further expected that the SE17 resistance gene provides full spectrum resistance to B1:1 to B1:36.

The majority of disease resistance genes in plants encode nucleotide-binding site leucine-rich repeat proteins, also known as NBS-LRR proteins (encoded by R genes). These proteins are characterized by nucleotide-binding site (NBS) and leucine-rich repeat (LRR) domains as well as variable amino- and carboxy-terminal domains and are involved in the detection of diverse pathogens, including bacteria, viruses, fungi, nematodes, insects and oomycetes. There are three major subfamilies of plant NBS-LRR proteins defined by the Toll/interleukin-1 receptor (TIR) also called TNLs, the coiled-coil (CC) motifs in the amino-terminal domain containing NBS-LRRs also called CNLs and RPW8-NLTRs also called RNLs. All these R genes contain a NB-ARC domain which is proposed to regulate activity of the R protein. The SE17 resistance gene comprises the region from an NB-ARC domain providing resistance to Bremia, represented by resistance domain 1. The NB-ARC domain is a functional ATPase domain, and its nucleotide-binding state is proposed to regulate activity of the R protein. The NB-ARC domain in R proteins likely functions as a molecular switch that, depending on the nucleotide bound, defines the activation state of the R protein.

The presence of the SE17 resistance gene will provide broad spectrum Bremia resistance to lettuce plants. To decrease the chances of the pathogen overcoming the resistance, as often seen with R genes, multiple R genes can be combined to enhance the durability of disease resistance. For example, the downy mildew resistant lettuce plant of the present invention may further comprise one or more resistance genes located at MRC2 (major resistance cluster 2) at a significant distance from the SE17 resistance gene or with R genes located at different linkage groups. As such, stacking of multiple resistance genes will enable broad and durable Bremia resistance in lettuce.

To demonstrate that the SE17 resistance gene provides Bremia resistance, this SE17 resistance gene was silenced by tobacco rattle virus (TRV)-based virus-induced gene silencing (VIGS) to induce susceptibility to B. lactucae infection in resistant L. serriola lettuce lines containing the resistance gene and L. sativa lines containing the SE17 resistance gene. With VIGS it was demonstrated that the SE17 resistance gene was associated with downy mildew resistance, since VIGS induced gene silencing was used to create Bremia susceptibility in resistant Lactuca accessions containing SE17. Resistant lettuce plants were transiently transformed with a silencing construct specific against the resistance SE17 gene which will result in the silencing of the resistance gene and as a consequence made the plant or plant organs susceptible to B. lactucae infection, thus by “removing” or silencing the SE17 resistance gene via virus induced gene silencing. To exclude that we do not target the previous identified Dm3 gene with VIGS (and are looking at allelic differences of Dm3), a VIGS control was included in the experiment which target the Dm3 gene which is also present in the MRC2 cluster (as is the SE17 gene of present invention).

According to another preferred embodiment, the present invention relates to the lettuce plant, wherein the one or more mutations in resistance domain 1 comprise at least a Glutamine (Q) to Arginine (R) amino acid substitution at position 24 (Q24R) and/or an Asparagine (N) to Serine (S) amino acid substitution at position 29 (N29S).

According to yet another preferred embodiment, the present invention relates to the Lettuce plant, wherein the one or more mutations in resistance domain 2 comprise at least a Threonine (T) to Isoleucine (I) amino acid substitution at position 104 (T104I) and/or a Threonine (T) to Asparagine (N) amino acid substitution at position 132 (T132N). Sequencing experiments showed that the protein encoded by the resistance conferring gene from the resistant plant comprises a further protein domain which differs in several amino acids that have been mutated, as compared with the corresponding protein encoded by the wild type SE17 gene of a plant that is susceptible.

According to yet another preferred embodiment, the present invention relates to the Lettuce plant, wherein resistance domain 1 is represented by the amino acid sequence of SEQ ID No. 4.

According to yet another preferred embodiment, the present invention relates to the Lettuce plant, wherein resistance domain 2 is represented by the amino acid sequence of SEQ ID No. 8.

According to yet another preferred embodiment, the present invention relates to the Lettuce plant, wherein the SE17 resistance gene encodes for a protein represented by the amino acid sequence of SEQ ID No 14.

According to yet another preferred embodiment, the present invention relates to the lettuce plant, wherein the plant is selected from Lactuca sativa, Lactuca virosa, Lactuca saligna, Lactuca serriola, Lactuca aculeate, Lactuca georgica, Lactuca perennis, Lactuca tatarica, Lactuca viminea, preferably Lactuca sativa.

According to a preferred embodiment, the present invention relates to the lettuce plant, wherein the one or more mutations are obtainable by genome editing techniques, preferably by mutagenesis (e.g. EMS), agrobacterium transformation and/or CRISPR/Cas techniques.

According to another preferred embodiment, the present invention relates to the lettuce plant, wherein the lettuce plant is further resistant to downy mildew caused by one or more of B. lactucae selected from the group of race B1:1 to B1:11. A lettuce plant of the present invention comprising the SE17 resistant gene is resistant to Bremia races from B1:12 to B1:36. Preferably, resistance to B. lactucae in the lettuce of present invention comprises full spectrum resistance to B. lactucae races B1:1 to B1:36. Disease resistance test show that the SE17 resistance gene further provides resistance to Bremia races B1:2, B1:4, B1:5, B1:10, and based on preliminary experiments it is expected that it provides full spectrum resistance to B1:1 to B1:36. According to a preferred embodiment, the present invention relates to the lettuce plant, wherein the SE17 resistance gene is at least heterozygously present in the lettuce plant, preferably homozygously.

According to yet another preferred embodiment, the present invention relates to the lettuce plant, wherein the SE17 resistance gene is obtainable, derived, or originates from a lettuce plant deposited under number NCIMB 43645.

According to another preferred embodiment, the present invention relates to the lettuce plant, wherein said lettuce plant comprises SEQ ID No. 9 and SEQ ID No. 10.

The present invention, according to a second aspect, relates to seed of a lettuce plant comprising a SE17 resistance gene encoding a protein comprising a resistance domain 1 represented by amino acid sequence of SEQ ID No. 2 and resistance domain 2 which is represented by the amino acid sequence of SEQ ID No. 6, wherein downy mildew resistance is provided by one or more mutations in resistance domain 1 and/or resistance domain 2. The seed comprises the SE17 resistance gene as described above.

The present invention, according to a third aspect, relates to a resistance gene, i.e. an SE17 resistance gene encoding a protein comprising a resistance domain 1 represented by amino acid sequence of SEQ ID No. 2 and resistance domain 2 with represented by the amino acid sequence of SEQ ID No. 6, wherein downy mildew resistance is provided by one or more mutations in resistance domain 1 and/or resistance domain 2. The SE17 resistant gene is a dominant trait.

According to a preferred embodiment, the present invention relates to a resistance gene that confers resistance to B. lactucae in lettuce plants, wherein the one or more mutations in a resistance domain 1 encoding the protein sequence represented by SEQ ID No. 2 comprises at least a Glutamine (Q) to Arginine (R) amino acid substitution at position 24 (Q24R) and/or a Asparagine (N) to Serine (S) amino acid substitution at position 29 (N29S), preferably at least both Q24R and N29S amino acid substitutions.

According to another preferred embodiment, the present invention relates to a resistance gene that confers resistance to B. lactucae in lettuce plants, wherein the one or more mutations in a resistance domain 2 encoding the protein sequence represented by SEQ ID No. 6 comprises at least Threonine (T) to Isoleucine (I) amino acid substitution at position 104 (T104I) and/or a Threonine (T) to Asparagine (N) amino acid substitution at position 132 (T132N), preferably at least both T104I and T132N amino acid substitutions.

According to yet another preferred embodiment, the present invention relates to a resistance gene that confers resistance to B. lactucae in lettuce plants, wherein the resistance gene comprises a resistance domain 1 that encodes for a protein comprising the sequence represented by SEQ ID No. 4.

According to another preferred embodiment, the present invention relates to a resistance gene that confers resistance to B. lactucae in lettuce plants, wherein the resistance gene comprises a resistance domain 2 that encodes for a protein comprising the sequence represented by SEQ ID No. 8.

According to another preferred embodiment, the present invention relates to the resistance gene that confers resistance to B. lactucae in lettuce plants, wherein resistance to B. lactucae in lettuce comprises resistance to B. lactucae of race B1:12 to B1:36. Preferably, the resistance spectrum to B. lactucae in lettuce comprises resistance to B. lactucae of B1:1 to B1:36. The resistance gene further provides resistance to B. lactucae US spectrum BL:1 to BL:9.

According to yet another preferred embodiment, the present invention relates to the resistance gene that confers resistance to B. lactucae in lettuce plants, wherein the plant is selected from Lactuca sativa, Lactuca virosa, Lactuca saligna, Lactuca serriola, Lactuca aculeate, Lactuca georgica, Lactuca perennis, Lactuca tatarica, Lactuca viminea, preferably Lactuca sativa.

According to yet another preferred embodiment, the present invention relates to the resistance gene that confers resistance to B. lactucae in lettuce plants, wherein the resistance gene is at least heterozygously present in the lettuce plant, preferably homozygously.

According to a preferred embodiment, the present invention relates to the resistance gene, wherein the protein is represented by amino acid sequence of SEQ ID No 14.

According to another preferred embodiment, the present invention relates to the resistance gene, wherein the SE17 resistance gene comprises SEQ ID No 13.

The present invention, according to a further aspect, relates to a method for identifying a downy mildew resistant lettuce plant of present invention, the method comprises the step of establishing, in the genome of a plant the presence of an SE17 resistance gene encoding a protein comprising a resistance domain 1 represented by amino acid sequence of SEQ ID No. 2 and resistance domain 2 with represented by the amino acid sequence of SEQ ID No. 6, wherein downy mildew resistance is provided by one or more mutations in resistance domain 1 and/or resistance domain 2, preferably SEQ ID No. 4 and/or. SEQ ID No. 8.

The present invention, according to a further aspect, relates to a method for identifying a downy mildew resistant lettuce plant of present invention, wherein the step of establishing, in the genome of a plant the presence of a SE17 resistance gene encoding a protein as, comprises establishing the presence of one or more sequences selected from the group consisting of SEQ ID No. 3, SEQ ID No. 7, SEQ ID No. 9, SEQ ID No. 10 or SEQ ID No. 13.

The present invention, according to a further aspect, relates to a method for obtaining a lettuce plant that is resistant to downy mildew, wherein the method comprises the steps of,
a) crossing a lettuce plant comprised of the resistance gene of the present invention with a lettuce plant susceptible to downy mildew and which does not comprise said resistance gene,
b) optionally, selfing the plant obtained in step a) for at least one time,
c) selecting the plants that are resistant to downy mildew.
   In the method of present invention, the lettuce plant is selected from Lactuca sativa, Lactuca virosa, Lactuca saligna, Lactuca serriola, Lactuca aculeate, Lactuca georgica, Lactuca perennis, Lactuca tatarica, Lactuca viminea, preferably Lactuca sativa.

The present invention, according to a further aspect, relates to a method for providing a lettuce plant that is resistant to downy mildew caused by B. lactucae race B1:12 to B1:36, wherein the method comprises the step of providing one or more mutations in a resistance domain 1 and/or resistance domain 2 encoding a protein sequence represented by SEQ ID No. 2 and/or SEQ ID No. 6, respectively, or having at least 98% sequence identity with SEQ ID No. 2 and/or SEQ ID No. 6.

According to a preferred embodiment, the present invention relates to a method, wherein the one or more mutations comprises a Glutamine (Q) to Arginine (R) amino acid substitution at position 24 (Q24R) and/or a Asparagine (N) to Serine (S) amino acid substitution at position 29 (N29S) in a resistance domain 1 that encodes for a protein comprising the sequence represented by SEQ ID No. 2, or having at least 98% sequence identity with SEQ ID No. 2, preferably both Q24R and N29S amino acid substitutions are present.

According to another preferred embodiment, the present invention relates to a method, wherein the one or more mutations further comprises a Threonine (T) to Isoleucine (I) amino acid substitution at position 104 (T104I) and/or a Threonine (T) to Asparagine (N) amino acid substitution at position 132 (T132N) in a resistance domain 2 that encodes for a protein comprising the sequence represented by SEQ ID No. 6, or having at least 98% sequence identity with SEQ ID No. 6, preferably both T104I and T132N amino acid substitutions are present.

According to a preferred embodiment, the present invention relates to the method, wherein the one or more mutations are provided by genome editing techniques, preferably by mutagenesis and/or CRISPR/Cas. The lettuce plant comprising the mutations in the SE17 resistance gene is selected from Lactuca sativa, Lactuca virosa, Lactuca saligna, Lactuca serriola, Lactuca aculeate, Lactuca georgica, Lactuca perennis, Lactuca tatarica, Lactuca viminea, preferably Lactuca sativa. A plant having this resistant phenotype can be obtained via use of gene editing and/or mutation techniques, such as EMS mutagenesis or CRISPR/Cas in concert with cloning techniques on the SE17 resistance gene, more specifically in domain 1 and/or 2 of SE17 resistance gene, to generate disease resistant crops. Mutations induced by gene editing techniques such as mutagenesis, CRISPR/Cas, transgenic techniques, or others can be regarded as non-natural mutations. Alternatively, a resistance gene can be brought into the plant by means of transgenic techniques or by introgression, wherein the mutated sequence(s) are being introduced into the plant.

The present invention, according to a further aspect, relates to the use of a gene construct or plasmid for introducing a resistance gene into the genome of a plant or plant cell and providing broad spectrum resistance to downy mildew caused by one or more of B. lactucae races selected from the group of race B1:12 to B1:36, wherein the gene construct is comprised of the resistance gene operably linked to expression providing sequences in said plant. The resistance gene of present invention may be transferred (e.g. by transformation or transfection) into plants, such as lettuce plants, using a plasmid or vector or linear gene construct that comprises the resistance gene of present invention. The SE17 resistance gene, after being transferred into the lettuce plant would provide resistance to B. lactucae, i.e. resistance to at least B. lactucae of race B1:2, B1:4, B1:5, B1:10, and B1:12 to B1:36, preferably B1:1 to B1:36.

The present invention will be further detailed in the following examples and figures wherein:

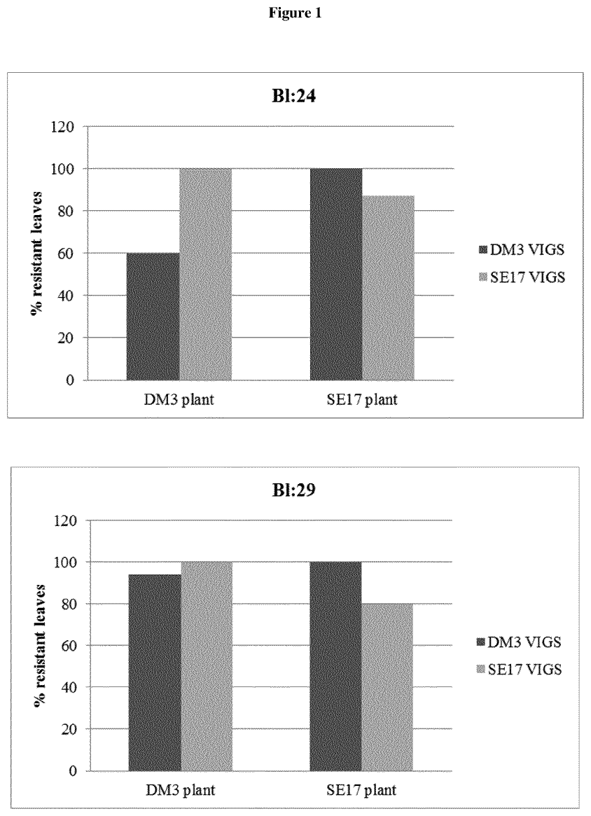

FIG. 1: shows the % of resistant leaves of Lettuce that have been infected with Bremia lactucae B1:24 or B129, after VIGS silencing of either the SE17 resistance gene of present invention or the DM3 resistance gene in a plant comprising the DM3 resistance gene (DM3 plant) or a plant of present invention comprising the SE17 resistance gene (SE17 plant). The SE17 or DM3 gene was silenced in these plants using VIGS gene silencing and subsequently infected with B. lactucae. In the samples with a resistant phenotype (DM3 or SE17 plants), there is no Bremia present at all, 100% resistant leaves for both B1:24 and B1:29. In the samples with susceptible phenotypes, Bremia is present resulting in lowering % of resistant leaves. As expected with transient gene silencing, VIGS gene silencing does not result in fully 100% silencing of the gene in all plants. However, the leaves from plants wherein the SE17 resistance gene has been silenced by VIGS silencing, showed a higher number of susceptible leaves when infected with Bremia as compared to plants where the SE17 gene was not silenced (i.e. by DM3 VIGS on the SE17 plant).

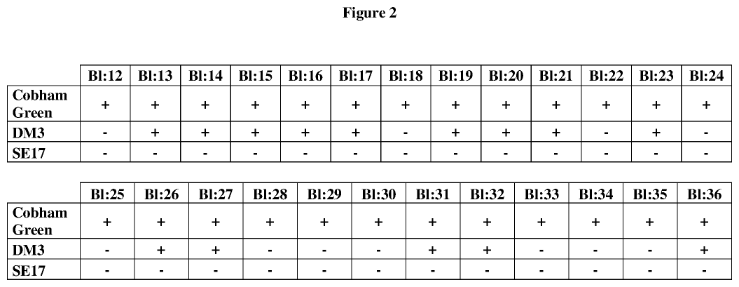

FIG. 2: shows an overview of the disease test performed with the most recent isolates of B. lactucae B1:12 to B1:36 on L. sativa lines Cobham Green R273, DM3 line, and the plant of present invention comprising the SE17 resistance gene. The plant of present invention shows to be resistant to all tested downy mildew isolates, B1:12 to B1:36, providing broad spectrum resistance.

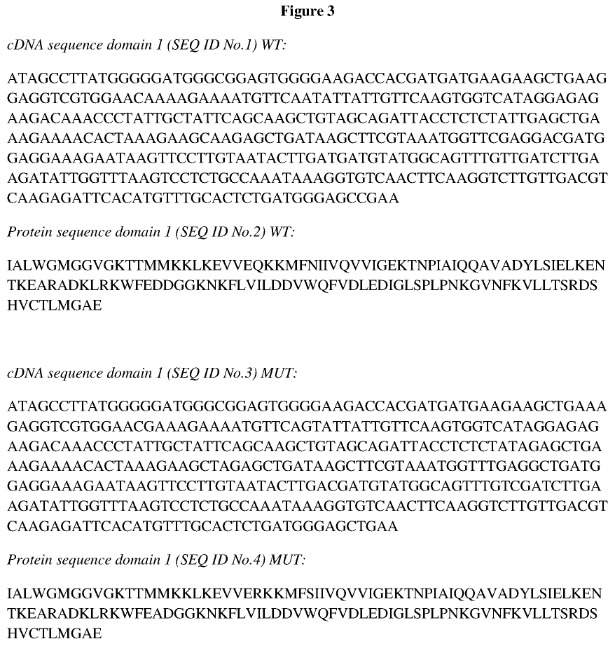

FIG. 3: shows the wild type (non mutated) cDNA sequence of domain 1 (SEQ ID No. 1) of the SE17 gene and the wild type protein sequence of domain 1 (SEQ ID No. 2) of L. sativa. Furthermore, the mutated cDNA sequence of domain 1 (SEQ ID No. 3) and of the SE17 gene and the mutated protein sequence of domain 1 (SEQ ID No. 4) comprising Q24R and N29S amino acid substitutions.

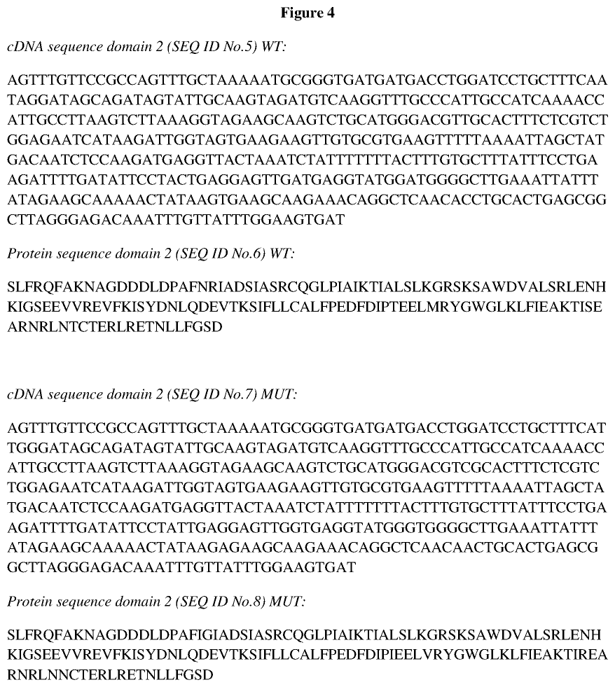

FIG. 4: shows the wild type (non mutated) cDNA sequence of domain 2 (SEQ ID No. 5) of the SE17 gene and the wild type protein sequence of domain 2 (SEQ ID No. 6) of L. sativa. Furthermore, the mutated cDNA sequence of domain 2 (SEQ ID No. 7) and of the SE17 gene and the mutated protein sequence of domain 2 (SEQ ID No. 8) comprising T104I and T132N amino acid substitutions.

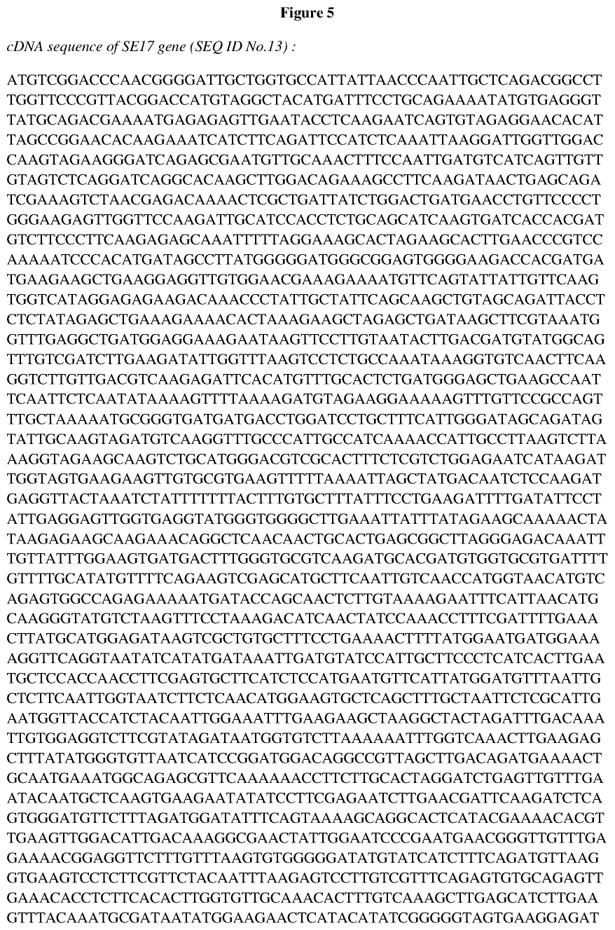

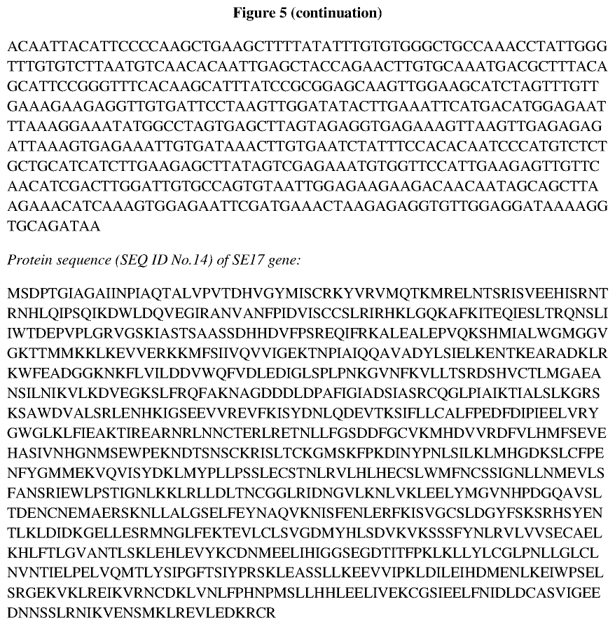

FIG. 5: shows the cDNA (SEQ ID No. 13) and protein sequence (SEQ ID No. 14) encoded by the SE17 gene of present invention providing Bremia (B. lactucae) resistance in lettuce.

### EXAMPLES

Gene Mapping RESISTANCE GENE Resistance Gene in L. serriola

Gene mapping experiments were done to identify a resistance gene that is involved in full spectrum Bremia (B. lactucae) resistance in lettuce (L. sativa). The resistance gene was originally isolated from L. serriola lettuce and was mapped on chromosome 2.

After fine mapping in a population of 12,000 plants there were several putative R genes present in the identified resistance locus. The identified resistance locus is flanked by two markers; the marker 1 (SEQ ID No. 9) and marker 2 (SEQ ID No. 10), providing a resistance locus of approximately 500.000 bp, which comprises several R genes, including the known DM3 resistance gene and a novel resistance gene identified as SE17.

| Marker | Sequence |
|---|---|
| Marker 1 (SEQ ID | TAATGGCTTACATGTGCCCAATCCATTCGTAATCGGCTCGGGTC |
| No. 9) | CTCCAGGGACCAACTATAAAGTCATGAAAAAAGCTTTCGTTGAA |
| | GGCTGGGGTGCAGTCATAGCTAAAATAGTAAGT |
| Marker 2 (SEQ ID | TGAAGATGTATACGAGGAGCCAGTTTTGGACATTGACAATGCTG |
| No. 10) | ATAAGGGTAATCCCCTGGCTGTGGTTGAGTACATTG |

VIGS silencing was used to silence the SE17 resistance gene in a resistant lettuce plant to confirm that this gene is needed for resistance and not the closely related to the known DM3 resistance gene, see below. These experiments indicated that when SE17 was silenced the plants became susceptible after Bremia infection. This confirms that the resistance gene is linked to a resistance gene that provides the plant resistance against Bremia.

Construction of Resistance Gene Construct and Transformation into Lettuce (L. serriola).

After gene mapping, candidate genes were identified and a quantitative trait locus was identified according to flanking markers that identified the SE17 resistance gene. To identify if this resistance gene was indeed responsible for the observed resistance, VIGS silencing was used to silence the resistance gene. Therefore, two VIGS-constructs were used, one that results in silencing of the SE17 resistance gene and one that silenced another known resistance gene present in same locus on MRC2 (major resistance cluster 2), i.e. DM3, that served as a control in the VIGS experiment to determine that the newly identified resistance gene is another gene than Dm3. The VIGS constructs were cloned in the K20 vector (See Table 1 for sequences, respectively SEQ ID No. 11, SEQ ID No. 12). The constructs were transformed and transiently expressed into a lettuce plant of present invention that is resistant to Bremia, using co-cultivation with agrobacterium (GV3101) to study the resistance gene function in relation to Bremia resistance. The % of resistant Bremia leaves was observed in both groups and both silencing constructs. With the leaves of VIGS-experiments independent disease tests (see below) were performed to observe that when SE17 resistance gene was silenced, plants became susceptible to Bremia.

TABLE 1

| VIGS-constructs | Sequence |
|---|---|
| SE17-VIGS | TGTTCATTAAGGATGTTTGATTGCTCTTCAATTGGTAATCTTCTC |
| (SEQ ID No. 11) | AACATGGAAGTGCTCAGCTTTGCTAATTCTAACATTGAATGGTT |
| | ACCATCTACAATTGGAAATTTGAAGAAGCTAAGGCTACTAGATT |
| | TGACAAATTGTAAAGGTCTTCGTATAGATAATGGTGTCTTAAAA |
| | AATTTGGTCAAACTTGAAGAGCTTTATATGGGTGTTAATCGTCC |
| | GTAT |
| DM3-VIGS | AGAATCTTGAACGATTCAAGATCTCAGTGGGATGCTCTTTTGAT |
| (SEQ ID No. 12) | GAAAATATCAATATGAGTAGCCACTCATACGAAAACATGTTGCA |
| | ATTGGTGACCAACAAAGGTGATGTATTAGACTCTAAACTTAATG |
| | GGTTATTTTTGAAAACAGAGGTGCTTTTTTTAAGTGTGCATGGCA |
| | TGAATGATCTTGAAGATGTTGAGGTGAAGTCGACACATCCTACT |
| | CAGTCCTCTTCATTCTGCAATTTAAAAGT |

### SE17 Resistance Gene Silencing Experiment Using Virus Induced Gene Silencing (VIGS)

Tobacco rattle virus (TRV)-derived VIGS vectors have been abundantly described to study gene function in Arabidopsis thaliana, Nicotiana benthamiana, Solanum esculentum and other plants (see for example Huang C, Qian Y, Li Z, Zhou X.: Virus-induced gene silencing and its application in plant functional genomics. Sci China Life Sci. 2012; 55(2):99-108).

Briefly, lettuce containing the SE17 resistance gene were silenced for SE17 resistance gene by VIGS. Also, the same experiments were performed for the DM3 gene to show that this gene is not contributing to resistance since it is present in the same resistance locus. Furthermore, independent of resistance gene silencing the PDS gene is silenced as well that serves as positive control to indicate if VIGS is working and to determine the efficiency. The PDS gene is involved in carotenoid biosynthesis and is the first step in lycopene biosynthesis. This step is catalyzed by the enzyme phytoene desaturase (PDS). When silencing of the PDS gene is achieved, this results in bleached leaves. Experiments showed bleached leaves indicating that the VIGS silencing was achieved and performed correctly (data not shown). All plants that were VIGS inoculated were harvested and put in a tray and sprayed with Bremia to test the effect of the gene silencing on disease resistance.

### Disease Test and Biotest for Downy Mildew in Lettuce

Leaves of resistant plants transiently transformed with the above described VIGS constructs, were put in trays with moistened paperboard and infected with Bremia race 24 or 29. B124 or BL29 infected seedlings are suspended in 20 mL water, filtered by cheesecloth and the flow-through is collected in a spray flask. The trays are spray-inoculated with the B. lactucae suspension. The trays are covered with a glass plate and stored in a climate chamber at 15° C. (12 hours of light). A black, opaque foil is placed over the trays for one day to improve growth of B. lactucae. After one day, the foil is removed. Experiments were performed in triple, and eight to ten days after infection leaves are phenotypically scored by eye on the presence of Bremia, i.e. being susceptible or resistant (FIG. 1).

Disease resistance tests show that the SE17 resistance gene provides resistance to Bremia races from B1:16 to B1:36 (See FIG. 2). Furthermore, disease resistance test show that the SE17 resistance gene further provides resistance to Bremia races B1:2, B1:4, B1:5, B1:10, and B1:12 to B1:15. It is expected that the SE17 resistance gene provides full spectrum resistance to B1:1 to B1:36.

A single gene line comprising the SE17 resistance gene was used internally to test Bremia diagnostic. Seeds of this line are deposited at NCIMB Ltd, Ferguson Building, Craibstone Estate, Bucksburn, Aberdeen AB21 9YA, Scotland on 5 Aug. 2020 under the number NCIMB 43645.

### Production of Downy Mildew Resistant Lettuce Plant Using Prime Editor (PE) System for Lettuce Protoplasts/Cotyledon Explants

We selected the SE17 resistance gene in lettuce to generate resistant plants of present invention comprising the Q24R/N29S double mutations in domain 1 by prime editing (PE). In a second experiment we mutated T104I/T132N in domain 2.

PE is a new CRISPR-Cas9 based gene-editing technology used for making specific mutations in the target genome. Prime editing can introduce any specific base change required, even small defined deletions or insertions, in a broader window. PE makes use of a SpCas9H840A nickase fused to a reverse transcriptase (RT) and a 3′ elongated guide RNA (pegRNA) carrying the desired mutations to obtain the mutated resistance gene in lettuce. This versatile pegRNA is a modified sgRNA that carries a reverse transcription template and primer binding site. This pegRNA anneals to the target locus and is used by the RT as a template to introduce the desired mutations into the genome of lettuce, as was described previously for plants (Lin et al., 2020, Nature Biotechnology, and Tang et al., 2020, Molecular Plant).

We made use of the PPE-V02 plasmid and used the sequences of the Cas9 (H840A), M-MLV RT with 3×NLS and atHSP terminator as described by Tang et al., plant codon optimized and re-synthesized commercially via Twist Bioscience (PPE: plant prime editor). The ZmUbi1 promotor was changed into the Lettuce Ubiquitin promotor (as described by Kawazu et al., 2019, The Horticulture Journal), to drive the Cas9H840A nickase. Compared with single guide RNAs (sgRNAs), pegRNAs have an additional 3′ extension composed of a primer binding site and a reverse-transcription template. To determine the best pegRNA sequence we made use of the web tool pegFinder as described previously (Chow et al., 2020, Nature Biomedical Engineering) (http://pegfinder.sidichenlab.org). Subsequently sequences of domain 1 of SE17 with and without the desired Q24R/N29S double mutations were selected. The top hit pegRNA was fused to the AtU6 promoter and synthesized commercially via Twist Bioscience. To construct the binary vector, the PPE and pegRNA cassette were cloned into the same backbone as described by Tang et al. (2020) via the ClonExpress II One Step Cloning Kit (Vazyme). The binary plasmid was transformed into Agrobacterium tumefaciens strain GV2260 and colonies were analyzed using PCR.

Next, lettuce cotyledon explants were transformed with the plasmid containing agrobacterium as described before (Sun et al., 2006, FEBS letters) followed by selection and regeneration. To detect targeted mutations, fragments spanning the target from genomic DNA were amplified and sequenced using the Illumina platform. Plants containing the desired mutant allele in either homozygous or heterozygous state were self-pollinated. In the following generation, plants were selected on the presence of the homozygous mutant allele and the absence of the transgene.

Similar to the mutations in domain 1 of SE17, prime editing technology was used to mutate domain 2 of SE17. In domain 2, the base pairs C to T (ACT to ATT) leading to T104I mutation and C to A (ACC to AAC) leading to T132N mutation were obtained.

These mutant plants were put in a Bremia test using a detached leaf assay and scored for disease symptoms. The expected resistant phenotype was observed an no Bremia disease symptoms were observed in the plants comprising the mutated domains.

1. A downy mildew resistant lettuce plant, wherein said lettuce plant comprises a SE17 resistance gene encoding a protein comprising a resistance domain 1 represented by amino acid sequence of SEQ ID No. 2 and resistance domain 2 with represented by the amino acid sequence of SEQ ID No. 6, wherein downy mildew resistance is provided by one or more mutations in resistance domain 1 and/or resistance domain 2 and wherein said lettuce plant is resistant to Bremia lactucae races B1:12 to B1:36.

2. Lettuce plant according to claim 1, wherein the one or more mutations in resistance domain 1 comprise at least a Glutamine (Q) to Arginine (R) amino acid substitution at position 24 (Q24R) and/or a Asparagine (N) to Serine (S) amino acid substitution at position 29 (N29S).

3. Lettuce plant according to claim 1 or 2, wherein the one or more mutations in resistance domain 2 comprise at least a Threonine (T) to Isoleucine (I) amino acid substitution at position 104 (T104I) and/or a Threonine (T) to Asparagine (N) amino acid substitution at position 132 (T132N).

4. Lettuce plant according to claim 2, wherein resistance domain 1 is represented by the amino acid sequence of SEQ ID No. 4.

5. Lettuce plant according to claim 3 wherein resistance domain 2 is represented by the amino acid sequence of SEQ ID No. 8.

6. Lettuce plant according to any one of the claims 1 to 5, wherein the SE17 resistance gene encodes for a protein represented by the amino acid sequence of SEQ ID No 14.

7. Lettuce plant according to any one of the claims 1 to 6, wherein the lettuce plant is selected from the group consisting of Lactuca sativa, Lactuca virosa, Lactuca saligna, Lactuca serriola, Lactuca aculeate, Lactuca georgica, Lactuca perennis, Lactuca tatarica, and Lactuca viminea.

8. Lettuce plant according to any one of the claims 1 to 7, wherein the lettuce plant is further resistant to one or more of Bremia lactucae races selected from the group consisting of races B1:1 to B1:11.

9. Lettuce plant according to any one of the claims 1 to 8, wherein the SE17 resistance gene is obtainable, derived, or originates from a lettuce plant deposited under number NCIMB 43645.

10. Seed of a lettuce plant according to any one of the claims 1 to 9, comprising a SE17 resistance gene encoding a protein as defined in any one of the claims 1 to 9.

11. SE17 resistance gene encoding a protein as defined in any one of the claims 1 to 10.

12. SE17 resistance gene according to claim 11, wherein the protein is represented by amino acid sequence of SEQ ID No 14.

13. SE17 resistance gene according to claim 11 or 12, wherein the SE17 resistance gene comprises SEQ ID No 13.

14. Method for identifying a downy mildew resistant lettuce plant according to any one of the claims 1 to 8, the method comprises the step of establishing, in the genome of a plant the presence of a SE17 resistance gene encoding a protein as defined in any one of the claims 1 to 9.

15. Method according to claim 14, wherein the step of establishing, in the genome of a plant the presence of a SE17 resistance gene encoding a protein as defined in any one of the claims 1 to 8 comprising establishing the presence of one or more sequences selected from the group consisting of SEQ ID No. 3, SEQ ID No. 7, SEQ ID No. 9, SEQ ID No. 10 or SEQ ID No. 13.

16. Method for providing a lettuce plant that is resistant to downy mildew, wherein the method comprises the steps of,
a) crossing a lettuce plant comprising a resistance gene according to any one of the claims 11 to 13 with a lettuce plant is susceptible to downy mildew and does not comprise said resistance gene,
b) optionally, selfing the plant obtained in step a) for at least one time,
c) selecting the plants that are resistant to downy mildew.

17. Use of a gene construct or plasmid for introducing a resistance gene into the genome of a plant or plant cell and providing broad spectrum resistance to downy mildew caused by one or more of B. lactucae selected from the group of races B1:12 to B1:36, wherein the gene construct is comprised of the resistance gene according to any one of the claims 11 to 13 operably linked to expression providing sequences in said plant.


SEQUENCE LISTING (14 sequences)

SEQ ID NO: 1 - DNA, 384 bp, Lactuca sativa
  wt
atagccttatgggggatgggcggagtggggaagaccacgatgatgaagaagctgaaggaggtcgtggaacaaaagaaaatgttcaatattattgttcaagtggtcataggagagaagacaaaccctattgctattcagcaagctgtagcagattacctctctattgagctgaaagaaaacactaaagaagcaagagctgataagcttcgtaaatggttcgaggacgatggaggaaagaataagttccttgtaatacttgatgatgtatggcagtttgttgatcttgaagatattggtttaagtcctctgccaaataaaggtgtcaacttcaaggtcttgttgacgtcaagagattcacatgtttgcactctgatgggagccgaa

SEQ ID NO: 2 - protein, 128 aa, Lactuca sativa
  wt
IALWGMGGVGKTTMMKKLKEVVEQKKMFNIIVQVVIGEKTNPIAIQQAVADYLSIELKENTKEARADKLRKWFEDDGGKNKFLVILDDVWQFVDLEDIGLSPLPNKGVNFKVLLTSRDSHVCTLMGAE

SEQ ID NO: 3 - DNA, 384 bp, Lactuca sativa
  mut
atagccttatgggggatgggcggagtggggaagaccacgatgatgaagaagctgaaagaggtcgtggaacgaaagaaaatgttcagtattattgttcaagtggtcataggagagaagacaaaccctattgctattcagcaagctgtagcagattacctctctatagagctgaaagaaaacactaaagaagctagagctgataagcttcgtaaatggtttgaggctgatggaggaaagaataagttccttgtaatacttgacgatgtatggcagtttgtcgatcttgaagatattggtttaagtcctctgccaaataaaggtgtcaacttcaaggtcttgttgacgtcaagagattcacatgtttgcactctgatgggagctgaa

SEQ ID NO: 4 - protein, 128 aa, Lactuca sativa
  mut
IALWGMGGVGKTTMMKKLKEVVERKKMFSIIVQVVIGEKTNPIAIQQAVADYLSIELKENTKEARADKLRKWFEADGGKNKFLVILDDVWQFVDLEDIGLSPLPNKGVNFKVLLTSRDSHVCTLMGAE

SEQ ID NO: 5 - DNA, 441 bp, Lactuca sativa
  wt
agtttgttccgccagtttgctaaaaatgcgggtgatgatgacctggatcctgctttcaataggatagcagatagtattgcaagtagatgtcaaggtttgcccattgccatcaaaaccattgccttaagtcttaaaggtagaagcaagtctgcatgggacgttgcactttctcgtctggagaatcataagattggtagtgaagaagttgtgcgtgaagtttttaaaattagctatgacaatctccaagatgaggttactaaatctatttttttactttgtgctttatttcctgaagattttgatattcctactgaggagttgatgaggtatggatggggcttgaaattatttatagaagcaaaaactataagtgaagcaagaaacaggctcaacacctgcactgagcggcttagggagacaaatttgttatttggaagtgat

SEQ ID NO: 6 - protein, 147 aa, Lactuca sativa
  wt
SLFRQFAKNAGDDDLDPAFNRIADSIASRCQGLPIAIKTIALSLKGRSKSAWDVALSRLENHKIGSEEVVREVFKISYDNLQDEVTKSIFLLCALFPEDFDIPTEELMRYGWGLKLFIEAKTISEARNRLNTCTERLRETNLLFGSD

SEQ ID NO: 7 - DNA, 441 bp, Lactuca sativa
  mut
agtttgttccgccagtttgctaaaaatgcgggtgatgatgacctggatcctgctttcattgggatagcagatagtattgcaagtagatgtcaaggtttgcccattgccatcaaaaccattgccttaagtcttaaaggtagaagcaagtctgcatgggacgtcgcactttctcgtctggagaatcataagattggtagtgaagaagttgtgcgtgaagtttttaaaattagctatgacaatctccaagatgaggttactaaatctatttttttactttgtgctttatttcctgaagattttgatattcctattgaggagttggtgaggtatgggtggggcttgaaattatttatagaagcaaaaactataagagaagcaagaaacaggctcaacaactgcactgagcggcttagggagacaaatttgttatttggaagtgat

SEQ ID NO: 8 - protein, 147 aa, Lactuca sativa
  mut
SLFRQFAKNAGDDDLDPAFIGIADSIASRCQGLPIAIKTIALSLKGRSKSAWDVALSRLENHKIGSEEVVREVFKISYDNLQDEVTKSIFLLCALFPEDFDIPIEELVRYGWGLKLFIEAKTIREARNRLNNCTERLRETNLLFGSD

SEQ ID NO: 9 - DNA, 121 bp, Artificial Sequence
  marker 1
taatggcttacatgtgcccaatccattcgtaatcggctcgggtcctccagggaccaactataaagtcatgaaaaaagctttcgttgaaggctggggtgcagtcatagctaaaatagtaagt

SEQ ID NO: 10 - DNA, 80 bp, Artificial Sequence
  marker 2
tgaagatgtatacgaggagccagttttggacattgacaatgctgataagggtaatcccctggctgtggttgagtacattg

SEQ ID NO: 11 - DNA, 225 bp, Artificial Sequence
  VIGS
tgttcattaaggatgtttgattgctcttcaattggtaatcttctcaacatggaagtgctcagctttgctaattctaacattgaatggttaccatctacaattggaaatttgaagaagctaaggctactagatttgacaaattgtaaaggtcttcgtatagataatggtgtcttaaaaaatttggtcaaacttgaagagctttatatgggtgttaatcgtccgtat

SEQ ID NO: 12 - DNA, 250 bp, Artificial Sequence
  VIGS
agaatcttgaacgattcaagatctcagtgggatgctcttttgatgaaaatatcaatatgagtagccactcatacgaaaacatgttgcaattggtgaccaacaaaggtgatgtattagactctaaacttaatgggttatttttgaaaacagaggtgctttttttaagtgtgcatggcatgaatgatcttgaagatgttgaggtgaagtcgacacatcctactcagtcctcttcattctgcaatttaaaagt

SEQ ID NO: 13 - DNA, 3027 bp, Lactuca sativa
  cDNA
atgtcggacccaacggggattgctggtgccattattaacccaattgctcagacggccttggttcccgttacggaccatgtaggctacatgatttcctgcagaaaatatgtgagggttatgcagacgaaaatgagagagttgaatacctcaagaatcagtgtagaggaacacattagccggaacacaagaaatcatcttcagattccatctcaaattaaggattggttggaccaagtagaagggatcagagcgaatgttgcaaactttccaattgatgtcatcagttgttgtagtctcaggatcaggcacaagcttggacagaaagccttcaagataactgagcagatcgaaagtctaacgagacaaaactcgctgattatctggactgatgaacctgttcccctgggaagagttggttccaagattgcatccacctctgcagcatcaagtgatcaccacgatgtcttcccttcaagagagcaaatttttaggaaagcactagaagcacttgaacccgtccaaaaatcccacatgatagccttatgggggatgggcggagtggggaagaccacgatgatgaagaagctgaaggaggttgtggaacgaaagaaaatgttcagtattattgttcaagtggtcataggagagaagacaaaccctattgctattcagcaagctgtagcagattacctctctatagagctgaaagaaaacactaaagaagctagagctgataagcttcgtaaatggtttgaggctgatggaggaaagaataagttccttgtaatacttgacgatgtatggcagtttgtcgatcttgaagatattggtttaagtcctctgccaaataaaggtgtcaacttcaaggtcttgttgacgtcaagagattcacatgtttgcactctgatgggagctgaagccaattcaattctcaatataaaagttttaaaagatgtagaaggaaaaagtttgttccgccagtttgctaaaaatgcgggtgatgatgacctggatcctgctttcattgggatagcagatagtattgcaagtagatgtcaaggtttgcccattgccatcaaaaccattgccttaagtcttaaaggtagaagcaagtctgcatgggacgtcgcactttctcgtctggagaatcataagattggtagtgaagaagttgtgcgtgaagtttttaaaattagctatgacaatctccaagatgaggttactaaatctatttttttactttgtgctttatttcctgaagattttgatattcctattgaggagttggtgaggtatgggtggggcttgaaattatttatagaagcaaaaactataagagaagcaagaaacaggctcaacaactgcactgagcggcttagggagacaaatttgttatttggaagtgatgactttgggtgcgtcaagatgcacgatgtggtgcgtgattttgttttgcatatgttttcagaagtcgagcatgcttcaattgtcaaccatggtaacatgtcagagtggccagagaaaaatgataccagcaactcttgtaaaagaatttcattaacatgcaagggtatgtctaagtttcctaaagacatcaactatccaaacctttcgattttgaaacttatgcatggagataagtcgctgtgctttcctgaaaacttttatggaatgatggaaaaggttcaggtaatatcatatgataaattgatgtatccattgcttccctcatcacttgaatgctccaccaaccttcgagtgcttcatctccatgaatgttcattatggatgtttaattgctcttcaattggtaatcttctcaacatggaagtgctcagctttgctaattctcgcattgaatggttaccatctacaattggaaatttgaagaagctaaggctactagatttgacaaattgtggaggtcttcgtatagataatggtgtcttaaaaaatttggtcaaacttgaagagctttatatgggtgttaatcatccggatggacaggccgttagcttgacagatgaaaactgcaatgaaatggcagagcgttcaaaaaaccttcttgcactaggatctgagttgtttgaatacaatgctcaagtgaagaatatatccttcgagaatcttgaacgattcaagatctcagtgggatgttctttagatggatatttcagtaaaagcaggcactcatacgaaaacacgttgaagttggacattgacaaaggcgaactattggaatcccgaatgaacgggttgtttgagaaaacggaggttctttgtttaagtgtgggggatatgtatcatctttcagatgttaaggtgaagtcctcttcgttctacaatttaagagtccttgtcgtttcagagtgtgcagagttgaaacacctcttcacacttggtgttgcaaacactttgtcaaagcttgagcatcttgaagtttacaaatgcgataatatggaagaactcatacatatcgggggtagtgaaggagatacaattacattccccaagctgaagcttttatatttgtgtgggctgccaaacctattgggtttgtgtcttaatgtcaacacaattgagctaccagaacttgtgcaaatgacgctttacagcattccgggtttcacaagcatttatccgcggagcaagttggaagcatctagtttgttgaaagaagaggttgtgattcctaagttggatatacttgaaattcatgacatggagaatttaaaggaaatatggcctagtgagcttagtagaggtgagaaagttaagttgagagagattaaagtgagaaattgtgataaacttgtgaatctatttccacacaatcccatgtctctgctgcatcatcttgaagagcttatagtcgagaaatgtggttccattgaagagttgttcaacatcgacttggattgtgccagtgtaattggagaagaagacaacaatagcagcttaagaaacatcaaagtggagaattcgatgaaactaagagaggtgttggaggataaaaggtgcagataa

SEQ ID NO: 14 - protein, 1008 aa, Lactuca sativa
MSDPTGIAGAIINPIAQTALVPVTDHVGYMISCRKYVRVMQTKMRELNTSRISVEEHISRNTRNHLQIPSQIKDWLDQVEGIRANVANFPIDVISCCSLRIRHKLGQKAFKITEQIESLTRQNSLIIWTDEPVPLGRVGSKIASTSAASSDHHDVFPSREQIFRKALEALEPVQKSHMIALWGMGGVGKTTMMKKLKEVVERKKMFSIIVQVVIGEKTNPIAIQQAVADYLSIELKENTKEARADKLRKWFEADGGKNKFLVILDDVWQFVDLEDIGLSPLPNKGVNFKVLLTSRDSHVCTLMGAEANSILNIKVLKDVEGKSLFRQFAKNAGDDDLDPAFIGIADSIASRCQGLPIAIKTIALSLKGRSKSAWDVALSRLENHKIGSEEVVREVFKISYDNLQDEVTKSIFLLCALFPEDFDIPIEELVRYGWGLKLFIEAKTIREARNRLNNCTERLRETNLLFGSDDFGCVKMHDVVRDFVLHMFSEVEHASIVNHGNMSEWPEKNDTSNSCKRISLTCKGMSKFPKDINYPNLSILKLMHGDKSLCFPENFYGMMEKVQVISYDKLMYPLLPSSLECSTNLRVLHLHECSLWMFNCSSIGNLLNMEVLSFANSRIEWLPSTIGNLKKLRLLDLTNCGGLRIDNGVLKNLVKLEELYMGVNHPDGQAVSLTDENCNEMAERSKNLLALGSELFEYNAQVKNISFENLERFKISVGCSLDGYFSKSRHSYENTLKLDIDKGELLESRMNGLFEKTEVLCLSVGDMYHLSDVKVKSSSFYNLRVLVVSECAELKHLFTLGVANTLSKLEHLEVYKCDNMEELIHIGGSEGDTITFPKLKLLYLCGLPNLLGLCLNVNTIELPELVQMTLYSIPGFTSIYPRSKLEASSLLKEEVVIPKLDILEIHDMENLKEIWPSELSRGEKVKLREIKVRNCDKLVNLFPHNPMSLLHHLEELIVEKCGSIEELFNIDLDCASVIGEEDNNSSLRNIKVENSMKLREVLEDKRCR